(19) United States
(12) Patent Application Publication
(10) Pub. No.: US 2026/0091135 A1
(43) Pub. Date: Apr. 2, 2026

(54) TREATMENT OF EPILEPSY

(71) Applicant: Modulight Bio Ltd., Ramat-Gan (IL)

(72) Inventors: Yotam ELDAR, Tel Aviv (IL); Yoav KFIR, Kiryat Tivon (IL)

(73) Assignee: Modulight Bio Ltd., Ramat-Gan (IL)

(21) Appl. No.: 19/403,102

(22) Filed: Nov. 27, 2025

Related U.S. Application Data
(63) Continuation of application No. PCT/IL2024/050531, filed on May 30, 2024.
(60) Provisional application No. 63/469,566, filed on May 30, 2023.

(51) Int. Cl. A61K 48/00; A61K 9/00; A61K 38/00; A61N 5/06; A61P 25/08; C07K 14/72; C12N 15/86

(52) U.S. Cl. CPC A61K 48/005; A61K 9/0085; A61K 48/0075; A61N 5/06; A61P 25/08; C07K 14/723; C12N 15/86; A61K 38/00; A61N 2005/0635; C07K 2319/01; C12N 2750/14143

(57) ABSTRACT

Methods of treating epilepsy are provided. Accordingly, there is provided a method of treating epilepsy in a subject in need thereof, the method comprising: (a) administering into a thalamus nucleus of the subject a therapeutically effective amount of a polynucleotide encoding a bistable type II opsin attached to a heterologous ER export signal and/or membrane trafficking signal which enables trafficking of said bistable type II opsin to axonal presynaptic terminals; and (b) exposing a neural region of said subject to light in a wavelength that activates said bistable type II opsin, wherein said neural region comprises a cell body and/or an axon of said thalamus nucleus.

### RELATED APPLICATIONS

This application is a Continuation of PCT Patent Application No. PCT/IL2024/050531 having International filing date of May 30, 2024 which claims the benefit of priority under 35 USC § 119(e) of U.S. Provisional Patent Application No. 63/469,566 filed on May 30, 2023. The contents of the above applications are all incorporated by reference as if fully set forth herein in their entirety.

### SEQUENCE LISTING STATEMENT

The XML file, entitled 104941SequenceListing.xml, created on Sep. 22, 2025, comprising 56,729 bytes, submitted concurrently with the filing of this application is incorporated herein by reference.

### FIELD AND BACKGROUND OF THE INVENTION

The present invention, in some embodiments thereof, relates to treatment of epilepsy.

Epilepsy is a neurological disorder characterized by recurrent, unprovoked seizures. It affects millions of individuals worldwide and poses significant challenges to their quality of life. Conventional treatments for epilepsy, such as antiepileptic drugs (AEDs) and surgical and ablation interventions, have limitations in terms of efficacy, adverse effects, and invasiveness. Deep-brain stimulation (DBS) has also been approved as a treatment modality for epilepsy (Salanova V. et al.; SANTÉ Study Group. (2021) Epilepsia. 62 (6): 1306-1317), albeit this treatment as well has limitations in terms of invasiveness and non-specific stimulation causing adverse effects.

In recent years, the development of cellular perturbation tools based on light sensitive proteins has resulted in a technology called optogenetics, referring to the integration of genetic and optical control to achieve gain- or loss-of-function of precisely defined events within specified cells of living tissues. This technique typically involves the use of proteins called opsins, a major class of light-sensitive proteins that can be found across all kingdoms of life and serve a diverse range of functions. Opsins can be divided into two groups, while both types are seven-transmembrane-domain proteins belonging to the G protein-coupled receptor (GPCR) superfamily, type I opsins (e.g. the microbial opsins) are ion channels or proton/ion pumps and thus are activated by light directly, while type II opsins activate G-proteins, which then activate effector enzymes that produce metabolites to e.g. open ion channels. Both types of opsins were suggested for optogenetic approaches (see e.g. International Patent Application Publication nos. WO2013090356 and WO2020/188572; Rost et al. Nat Neurosci (2022) 25 (8): 984-998; and Paz et al. Nat Neurosci (2013) 16 (1): 64-70).

### SUMMARY OF THE INVENTION

According to an aspect of some embodiments of the present invention there is provided a method of treating epilepsy in a subject in need thereof, the method comprising:
(a) administering into a thalamus nucleus of the subject a therapeutically effective amount of a polynucleotide encoding a bistable type II opsin attached to a heterologous ER export signal and/or membrane trafficking signal which enables trafficking of the bistable type II opsin to axonal presynaptic terminals; and
(b) exposing a neural region of the subject to light in a wavelength that activates the bistable type II opsin, wherein the neural region comprises a cell body and/or an axon of the thalamus nucleus,
thereby treating epilepsy in the subject.

According to some embodiments of the invention, the epilepsy is a generalized onset epilepsy.

According to some embodiments of the invention, the generalized onset epilepsy is selected from the group consisting of Developmental Epilepsy & Encephalopathy (DEE), drug-resistant generalized epilepsy, Lennox-Gastaut Syndrome (LGS), Dravet syndrome, epilepsy associated with a mutation in Slc6a1, epilepsy associated with a mutation in WWOX, generalized epilepsy of unknown origin, multifocal epilepsy, Absence epilepsy and Rasmussen's syndrome.

According to some embodiments of the invention, the generalized onset epilepsy is selected from the group consisting of drug-resistant generalized epilepsy, Lennox-Gastaut Syndrome (LGS), Dravet syndrome, epilepsy associated with a mutation in Slc6a1, epilepsy associated with a mutation in WWOX, generalized epilepsy of unknown origin, multifocal epilepsy and Absence epilepsy.

According to some embodiments of the invention, the epilepsy is a focal onset epilepsy.

According to some embodiments of the invention, the focal onset epilepsy is selected from the group consisting of drug-resistant focal epilepsy, temporal lobe epilepsy, frontal lobe epilepsy, parietal lobe epilepsy, Sturge Weber syndrome, Tuberous Sclerosis Complex, post-stroke epilepsy, post TBI epilepsy, focal cortical dysplasia, occipital lobe epilepsy and Epilpesia Partialis Continua (EPC).

According to some embodiments of the invention, the focal onset epilepsy is selected from the group consisting of drug-resistant focal epilepsy, temporal lobe epilepsy, frontal lobe epilepsy, parietal lobe epilepsy, Sturge Weber syndrome, Tuberous Sclerosis Complex, post-stroke epilepsy, post TBI epilepsy and focal cortical dysplasia.

According to some embodiments of the invention, the focal onset epilepsy comprises secondary generalized seizures.

According to some embodiments of the invention, the administering is by a stereotactic injection.

According to some embodiments of the invention, the thalamus nucleus is selected from the group consisting of Anterior Nucleus of the Thalamus (ANT), Intralaminar nucleus, lateral thalamic nucleus, medial thalamic nucleus, Centromedian Nucleus (CM), Ventral Posterior Nucleus (VB), Ventral Medial Nucleus (VM), Central Lateral (CL) and Pulvinar.

According to some embodiments of the invention, the thalamus nucleus is selected from the group consisting of Anterior Nucleus of the Thalamus (ANT), Intralaminar nucleus, lateral thalamic nucleus, medial thalamic nucleus, Centromedian Nucleus (CM), Ventral Posterior Nucleus (VB), Ventral Medial Nucleus (VM) and Pulvinar.

According to some embodiments of the invention, the polynucleotide is packed in a viral vector.

According to some embodiments of the invention, a nucleic acid sequence encoding the bistable type II opsin is codon optimized to mammalian expression.

According to some embodiments of the invention, the bistable type II opsin is selected from the group consisting of OPN3, OPN4, OPN5, LcPP, DrPP2, TrPP2, parapinopsin, PdCO, TMT and peropsin.

According to some embodiments of the invention, the bistable type II opsin is OPN3.

According to some embodiments of the invention, the OPN3 is mosquito OPN3 (MosOpn3).

According to some embodiments of the invention, the ER export signal and/or the membrane trafficking signal is of a protein expressed in neuronal cells.

According to some embodiments of the invention, the ER export signal and/or the membrane trafficking signal is of a Kir2.1 polypeptide.

According to some embodiments of the invention, an amino acid sequence of the ER export signal comprises SEQ ID NO: 2.

According to some embodiments of the invention, an amino acid sequence of the membrane trafficking signal comprises SEQ ID NO: 1.

According to some embodiments of the invention, the exposing is effected at least 6 weeks following the administering.

According to some embodiments of the invention, the exposing is effected at least 8 weeks following the administering.

According to some embodiments of the invention, the exposing comprises repeated illumination independent of detection of an epileptic seizure.

According to some embodiments of the invention, the exposing is effected upon detection of an epileptic seizure.

According to specific embodiments of the invention, the detection is by electrodes (e.g. external, epidural, subdural, or depth electrodes)

According to some embodiments of the invention, the detection is by EEG.

According to some embodiments of the invention, the exposing is effecting using a skull-mounted device.

According to some embodiments of the invention, the skull-mounted device allows electrophysiological recording and illumination.

According to some embodiments of the invention, the skull-mounted device allows EEG recording and illumination.

According to some embodiments of the invention, the wavelength is 450-650 nm.

According to some embodiments of the invention, the wavelength is 350-670 nm.

According to some embodiments of the invention, the neural region comprises a thalamus region.

According to some embodiments of the invention, the neural region comprises a cortex region.

According to some embodiments of the invention, the neural region comprises a presynaptic terminal of the axon.

1. A method of treating epilepsy in a subject in need thereof, the method comprising:
(a) administering into a thalamus nucleus of the subject a therapeutically effective amount of a polynucleotide encoding a bistable type II opsin attached to a heterologous ER export signal and/or membrane trafficking signal which enables trafficking of said bistable type II opsin to axonal presynaptic terminals; and
(b) exposing a neural region of said subject to light in a wavelength that activates said bistable type II opsin, wherein said neural region comprises a cell body and/or an axon of said thalamus nucleus,
thereby treating epilepsy in the subject.

2. A method of treating epilepsy in a subject in need thereof having been administered into a a thalamus nucleus a therapeutically effective amount of a polynucleotide encoding a bistable type II opsin attached to a heterologous ER export signal and/or membrane trafficking signal which enables trafficking of said bistable type II opsin to axonal presynaptic terminals, the method comprising exposing a neural region of said subject to light in a wavelength that activates said bistable type II opsin, wherein said neural region comprises a cell body and/or an axon of said thalamus nucleus, thereby treating epilepsy in the subject.

3. A composition comprising a polynucleotide encoding a bistable type II opsin attached to a heterologous ER export signal and/or membrane trafficking signal which enables trafficking of said bistable type II opsin to axonal presynaptic terminals, for use in treating epilepsy in a subject in need thereof, the composition being used in combination with a device for exposing a neural region of said subject to light, wherein the treatment is characterized by:
(a) administration of said composition into a thalamus nucleus of said subject; and
(b) exposure of the neural region of said subject to light in a wavelength that activates said bistable type II opsin, wherein said neural region comprises a cell body and/or an axon of said thalamus nucleus.

4. A composition comprising a polynucleotide encoding a bistable type II opsin attached to a heterologous ER export signal and/or membrane trafficking signal which enables trafficking of said bistable type II opsin to axonal presynaptic terminals, for use in treating epilepsy in a subject in need thereof, wherein the composition is formulated for administration to the thalamus.

5. The method of any one of claims 1-2 or the composition for use of any one of claims 3-4, wherein said epilepsy is a generalized onset epilepsy.

6 The method or composition for use of claim 5, wherein said generalized onset epilepsy is selected from the group consisting of Developmental Epilepsy & Encephalopathy (DEE), drug-resistant generalized epilepsy, Lennox-Gastaut Syndrome (LGS), Dravet syndrome, epilepsy associated with a mutation in Slc6a1, epilepsy associated with a mutation in WWOX, generalized epilepsy of unknown origin, multifocal epilepsy, Absence epilepsy and Rasmussen's syndrome.

7 The method of any one of claims 1-2 or the composition for use of any one of claims 3-4, wherein said epilepsy is a focal onset epilepsy.

8. The method or composition for use of claim 7, wherein said focal onset epilepsy is selected from the group consisting of drug-resistant focal epilepsy, temporal lobe epilepsy, frontal lobe epilepsy, parietal lobe epilepsy, Sturge Weber syndrome, Tuberous Sclerosis Complex, post-stroke epilepsy, post TBI epilepsy, focal cortical dysplasia, occipital lobe epilepsy and Epilpesia Partialis Continua (EPC).

9. The method or composition for use of any one of claims 7-8, wherein said focal onset epilepsy comprises secondary generalized seizures.

10. The method or composition for use of any one of claims 1-9, wherein said administering or said administration is by a stereotactic injection.

11. The method or composition for use of any one of claims 1-10, wherein said thalamus nucleus is selected from the group consisting of Anterior Nucleus of the Thalamus (ANT), Intralaminar nucleus, lateral thalamic nucleus, medial thalamic nucleus, Centromedian Nucleus (CM), Ventral Posterior Nucleus (VB), Ventral Medial Nucleus (VM), Central Lateral (CL) and Pulvinar.

12. The method or composition for use of any one of claims 1-11, wherein said polynucleotide is packed in a viral vector.

13. The method or composition for use of any one of claims 1-12, wherein a nucleic acid sequence encoding said bistable type II opsin is codon optimized to mammalian expression.

14. The method or composition for use of any one of claims 1-13, wherein said bistable type II opsin is selected from the group consisting of OPN3, OPN4, OPN5, LcPP, DrPP2, TrPP2, parapinopsin, PdCO, TMT and peropsin.

15. The method or composition for use of any one of claims 1-13, wherein said bistable type II opsin is OPN3.

16. The method or composition for use of claim 15, wherein said OPN3 is mosquito OPN3 (MosOpn3).

17. The method or composition for use of any one of claims 1-16, wherein said ER export signal and/or said membrane trafficking signal is of a protein expressed in neuronal cells.

18. The method or composition for use of any one of claims 1-17, wherein said ER export signal and/or said membrane trafficking signal is of a Kir2.1 polypeptide.

19. The method or composition for use of any one of claims 1-18, wherein an amino acid sequence of said ER export signal comprises SEQ ID NO: 2.

20 The method or composition for use of any one of claims 1-19, wherein an amino acid sequence of said membrane trafficking signal comprises SEQ ID NO: 1.

21. The method or composition for use of any one of claims 1-3 and 5-20, wherein said exposing is effected at least 6 weeks following said administering.

22. The method or composition for use of any one of claims 1-3 and 5-20, wherein said exposing is effected at least 8 weeks following said administering.

23. The method or the composition for use of any one of claims 1-3 and 5-22, wherein said exposing comprises repeated illumination independent of detection of an epileptic seizure.

24. The method or the composition for use of any one of claims 1-3 and 5-22, wherein said exposing is effected upon detection of an epileptic seizure.

25. The method or the composition for use of claim 24, wherein said detection is by an electrode.

26. The method of any one of claims 1-2 and 5-25, wherein said exposing is effecting using a skull-mounted device.

27. The composition for use of any one of claims 3 and 5-24, wherein said device is a skull-mounted device.

28. The method of claim 26 or the composition for use of claim 27, wherein said skull-mounted device allows electrophysiological recording and illumination.

29. The method or the composition for use of any one of claims 1-3 and 5-28, wherein said wavelength is 450-650 nm.

30. The method or the composition for use of any one of claims 1-3 and 28, wherein said wavelength is 350-670 nm.

31. The method or the composition for use of any one of claims 1-3 and 5-30, wherein said neural region comprises a thalamus region.

32. The method or the composition for use of any one of claims 1-3 and 5-30, wherein said neural region comprises a cortex region.

33. The method or the composition for use of any one of claims 1-3 and 5-32, wherein said neural region comprises a presynaptic terminal of said axon.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of an experimental timeline.

FIG. 2 is a schematic representation of the eOPN3-encoding AAVs injection site, specifically, the VB nucleus of the thalamus.

FIG. 3 is a schematic detailed representation of illumination and electrophysiological recording devices placement in the cortex (cortical region). The figure shows an optical fiber placed just above the cortex, and 2 depth electrodes that are inserted to layers 4 & 5 of the cortex, in order to record electrophysiological neural activity.

FIG. 4 is a schematic representation of the illumination target (cortex, indicating the various layers of the cortex) in reference to the injection site (specifically, the VB nucleus of the thalamus).

FIGS. 5A-B show representative images of histology slices from a mouse brain after injection of eOPN3-encoding AAVs. FIG. 5A depicts a full brain slice showing eOPN3 expression in the thalamus (site of injection) as well as cortical expression (axon projections from injection site). FIG. 5B depicts a zoom-in on the cortical region (white box in FIG. 5A), indicating the various layers of the cortex. The images were obtained by confocal microscopy utilizing the bio-fluorescent protein mScarlet encoded in the AAVs together with eOPN3.

FIG. 6 demonstrates cessation of PTZ-induced generalized seizure by illumination of cortical projections of VB thalamic neurons expressing eOPN3. Illumination lasted for 20 seconds at 10 mW intensity (green light, 525 nm). Mode of detection is detailed in FIGS. 3-4—EEG screws+cortical depth electrodes at layers 4 and 5 of the cortex. The effect lasted for approx. 6 minutes following the illumination.

### DESCRIPTION OF SPECIFIC EMBODIMENTS OF THE INVENTION

The present invention, in some embodiments thereof, relates to treatment of epilepsy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst reducing specific embodiments of the present invention to practice, the present inventors have now uncovered that bistable type II opsins can be used as a treatment modality for epilepsy, while utilizing the thalamic-cortical projections.

Thus, according to an aspect of the present invention, there is provided a method of treating epilepsy in a subject in need thereof, the method comprising:
(a) administering into a thalamus nucleus of the subject a therapeutically effective amount of a polynucleotide encoding a bistable type II opsin attached to a heterologous ER export signal and/or membrane trafficking signal which enables trafficking of said bistable type II opsin to axonal presynaptic terminals; and
(b) exposing a neural region of said subject to light in a wavelength that activates said bistable type II opsin, wherein said neural region comprises a cell body and/or an axon of said thalamus nucleus,
thereby treating epilepsy in the subject.

According to an additional or an alternative aspect of the present invention, there is provided a method of treating epilepsy in a subject in need thereof having been administered into a thalamus nucleus a therapeutically effective amount of a polynucleotide encoding a bistable type II opsin attached to a heterologous ER export signal and/or membrane trafficking signal which enables trafficking of said bistable type II opsin to axonal presynaptic terminals, the method comprising exposing a neural region of said subject to light in a wavelength that activates said bistable type II opsin, wherein said neural region comprises a cell body and/or an axon of said thalamus nucleus, thereby treating epilepsy in the subject.

According to an additional or an alternative aspect of the present invention, there is provided a composition comprising a polynucleotide encoding a bistable type II opsin attached to a heterologous ER export signal and/or membrane trafficking signal which enables trafficking of said bistable type II opsin to axonal presynaptic terminals, for use in treating epilepsy in a subject in need thereof, the composition being used in combination with a device for exposing a neural region of said subject to light, wherein the treatment is characterized by:
(a) administration of said composition into a thalamus nucleus of said subject; and
(b) exposure of the neural region of said subject to light in a wavelength that activates said bistable type II opsin, wherein said neural region comprises a cell body and/or an axon of said thalamus nucleus.

According to an additional or an alternative aspect of the present invention, there is provided a composition comprising a polynucleotide encoding a bistable type II opsin attached to a heterologous ER export signal and/or membrane trafficking signal which enables trafficking of said bistable type II opsin to axonal presynaptic terminals, for use in treating epilepsy in a subject in need thereof, wherein the treatment is characterized by:
(a) administration of said composition into thalamus nucleus of said subject; and
(b) activation of said bistable type II opsin by light.

According to an additional or an alternative aspect of the present invention, there is provided a composition comprising a polynucleotide encoding a bistable type II opsin attached to a heterologous ER export signal and/or membrane trafficking signal which enables trafficking of said bistable type II opsin to axonal presynaptic terminals, for use in treating epilepsy in a subject in need thereof, wherein the composition is formulated for administration to the thalamus.

As used herein, the term “treating” or “treatment” refers to inhibiting, preventing or arresting the development of a pathology (disease, disorder or medical condition i.e. epilepsy) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology, as further described infra.

According to specific embodiments, treating is manifested by arresting or inhibition of seizures.

According to specific embodiments, treating is manifested by preventing occurrence of seizures.

As used herein, the term “subject” includes mammals, e.g., human beings at any age and of any gender who suffers from the pathology (disease, disorder or medical condition i.e. epilepsy).

According to specific embodiments, the subject is a human subject.

According to specific embodiments, the subject is resistant to treatment with epileptic drugs or suffers from adverse effects associated with epileptic drugs that prevent their use.

However, according specific embodiments, the methods disclosed herein can be used in combination with other established or experimental therapeutic regimen to treat epilepsy including, but not limited to, surgical and ablation interventions, neuromodulation options, ketogenic dietary therapy and antiepileptic drugs (e.g. Brivaracetam, Clobazam, Felbamate, Lmotrigine, Levetiracetam, Perampanel, Rufinamide, Topiramate, Valproate, Zonisamine, Carbamazepine, Cenobamate, Gabapentin, Oxcarbazepine, Phenobarbital, Phenytoin, Pregabalin, Primidone, Stiripentol, Tiagabine, Vigabatrin etc.), which are well known in the art and disclosed for example in the Uptodate website: Chapter “evaluation and management of drug resistant epilepsy” by Joseph I Sirven, MD.

As used herein, the term “epilepsy” refers to a neurological disorder characterized by the presence of recurrent unprovoked seizures. Epileptic seizures are sudden and abnormal electrical disturbances in the brain, manifested by a wide range of symptoms, including loss of consciousness, convulsions, sensory disturbances, and altered behavior. Three main types of epilepsy are known: generalized onset epilepsy, focal onset epilepsy and unknown onset epilepsy.

Diagnosing epilepsy typically involves a comprehensive evaluation by a neurologist. It is primarily based on a combination of medical history, clinical examination and various diagnostic tests e.g. a neurological exam, neuropsychological tests, blood tests, genetic testing, electroencephalogram (EEG), brain imaging tests such as computerized tomography (CT) scan, magnetic resonance imaging (MRI), Positron emission tomography (PET). It is important to note that epilepsy diagnosis is often a process of elimination, as it involves ruling out other potential causes of seizures (e.g. head injuries, toxins, tumors, and infections). To be categorized as having epilepsy, a subject must experience two or more unprovoked seizures.

Non-limiting examples of measures that can be used to assess severity of epilepsy include seizure frequency, seizure duration, associated symptoms, quality of life impairment, etc.

According to specific embodiments, the epilepsy is generalized onset epilepsy.

Subjects with generalized epilepsy have generalized seizures which affect both hemispheres of the brain simultaneously or from the onset. Non-limiting examples of generalized onset epilepsy include Developmental Epilepsies & Encephalopathies (DEEs), Lennox-Gastaut Syndrome (LGS), Dravet syndrome, epilepsy associated with a mutation in Slc6a1, Rasmussen's syndrome, epilepsy associated with a mutation in WWOX, generalized epilepsy of unknown origin, multifocal epilepsy and Absence epilepsy. The seizures may be either motor (i.e. involving physical movement) or non-motor (do not involve physical movement). Some non-limiting motor symptoms include jerking movements, weakness or limp limbs, tense, rigid muscles, muscle twitching, full-body epileptic spasms. Some non-limiting non-motor symptoms include staring into space, a sudden stop in movement, brief twitches, fluttering eyelids, amnesia, difficulty in speaking.

According to specific embodiments, the epilepsy is focal onset epilepsy.

Subjects with focal epilepsy have focal seizures, which originate from a specific region or focus within one hemisphere of the brain. One non-limiting common example of focal onset epilepsy is temporal lobe epilepsy, where seizures typically arise from the temporal lobe of the brain. Other examples include, but not limited to, frontal lobe epilepsy, parietal lobe epilepsy, focal cortical dysplasia, occipital lobe epilepsy, Sturge Webber Syndrome, Tuberous Sclerosis complex, Epilepsia Partialis Continua (EPC), post-stroke epilepsy, post TBI epilepsy. According to specific embodiments, the focal onset epilepsy comprises secondary generalized seizures. Such seizures initially start as focal seizures but then spread and involve both hemispheres of the brain, leading to a generalized seizure (i.e. begins in a specific region but subsequently affects the entire brain). The seizures may be either motor or non-motor. Some non-limiting motor symptoms include muscle twitching, jerking, spasms, repeated movements, like clapping or chewing. Some non-limiting non-motor symptoms include waves of hot or cold, goosebumps, lack of movement, changes in emotions or thoughts.

According to specific embodiments, the epilepsy is unknown epilepsy (also known as unknown onset epilepsy, or epilepsy of unknown origin).

If the neurologists do not know where seizures originate, they will diagnose a subject with unknown epilepsy. Some non-limiting motor symptoms include stiffening and loss of consciousness, rapid, rhythmic jerking and convulsing, bluish face from lack of oxygen, loss of bladder and/or bowel control. Some non-limiting non-motor symptoms include a sudden stop in movement, vacant staring, stillness.

According to specific embodiments, the epilepsy is an intractable epilepsy (i.e. insensitive to available antiepileptic drugs, also referred to as drug-resistant epilepsy). Non-limiting forms of intractable epilepsy may be those that are caused by Malformations of Cortical Developments (MCD), Hippocampal Sclerosis (HS), or Sturge-Weber Syndrome (SWS), such as Focal Cortical Dysplasia (FCD), Hemimegaloencephaly (HME), and Tuberous Sclerosis Complex (TSC), as well as DEEs such as LGS, Dravet, Rasmussen's and other syndromes.

In some cases or subjects the seizures can begin with an “aura”, a subjective sensation or minor symptoms signifying the seizure's onset. This can feel like an uneasy feeling in the stomach (similar to the feeling of riding a rollercoaster), a certain smell or taste and the like.

The methods disclosed herein include administering into a thalamus nucleus of the subject a polynucleotide encoding a bistable type II opsin attached to a heterologous ER export signal and/or membrane trafficking signal. Such polynucleotides are disclosed for example in International Patent Application Publication No. WO2020/188572, the contents of which are fully incorporated herein by reference, and are further described hereinbelow.

Non-limiting examples of thalamus nuclei that can be used with specific embodiments of the invention include the Anterior Nucleus of the Thalamus (ANT), the Intralaminar nuclei, the lateral thalamic nuclei, the medial thalamic nuclei, the Centromedian Nucleus (CM), the CentroLateral (CL) nucleus, the Ventral Posterior Nucleus (VB), the Ventral Medial Nucleus (VM) and the Pulvinar.

Typically, to allow direct administration to the thalamus of a subject, the polynucleotide is administered in a local manner.

Thus, according to specific embodiments, the administering or administration is by a stereotactic injection.

As used herein, the term “stereotactic injection” refers to a technique for delivering a substance or medication to a precise and predetermined target site within the brain (e.g. thalamus nucleus). It involves the use of three-dimensional coordinates to guide the placement of a needle or catheter with high accuracy. According to specific embodiments, the stereotactic injection is MRI guided. The stereotactic injection allows for targeted delivery of the substance to the desired location, minimizing damage to surrounding tissues and optimizing therapeutic efficacy (e.g. minimizing off-target effects and allowing the use of smaller tiers and dosages, making the treatment safer and cheaper).

A Type II opsin is a G-coupled protein receptor (GPCR) which is made light-sensitive with an attached chromophore molecule that allows it to absorb light. Most type II opsins bind 11-cis retinal as a chromophore to form a photosensitive pigment (opsin-based pigment). The isomerization of the chromophore (e.g. 11-cis to all-trans) in an opsin-based pigment upon light absorption triggers G protein activation.

According to specific embodiments, the Type II opsin activates Gᵢ-type and Gₒ-type G protein in a light dependent manner.

According to specific embodiments, the Type II opsin activates G_{z}-type G protein in a light dependent manner.

Type II opsins do not comprise an ion channel or a proton/ion pump.

As used herein, the phrase “bistable type II opsin” refers to a type II opsin which remains bound to the chromophore (e.g. retinal) following illumination (i.e. does not undergo bleaching).

Hence, a bistable type II opsin displays prolonged signal transduction following a single illumination pulse. Typically, the bistable type II opsin reverts to an original dark state through thermal relaxation after minutes in the dark or by illumination with light at a different wavelength. Methods of determining bistability of the opsin are well known in the art and include spectroscopic measurements.

According to specific embodiments, the bistable type II opsin is a naturally occurring bistable type II opsin. Such naturally occurring bistable type II opsins are known in the art and include, but are not limited to OPN3 (e.g. MosOpn3), OPN4, OPN5, parapinopsin (e.g. LcPP, zPP1, pPP2, zPP2/DrPP2, pPP2/TrPP2), PdCO (e.g. PdCO2), TMT (e.g. PufTMT, medakaTMT1A), peropsin.

According to specific embodiments, the bistable type II opsin is selected from the group consisting of OPN3, OPN4, OPN5, parapinopsin, zPP2, pPP2, PdCO, TMT and peropsin

According to specific embodiments, the bistable type II opsin is selected from the group consisting of OPN3, OPN4, OPN5, parapinopsin, PdCO and peropsin.

According to specific embodiments, the bistable type II opsin is selected from the group consisting of OPN3, parapinopsin, PdCO and TMT.

According to specific embodiments, the bistable type II opsin is selected from the group consisting of OPN3, parapinopsin and PdCO.

Any of the bistable type II opsins disclosed herein also encompass functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the sequences of the wild type opsins disclosed herein (as also exemplified by specific accession numbers and amino acid sequences); or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution, as further described hereinbelow.

According to specific embodiments, the opsin may comprise conservative and non-conservative amino acid substitutions.

According to specific embodiments, the bistable type II opsin activates Gio signaling in a cell expressing same following exposure to light in a wavelength that activates it, as determined by e.g. GsX assay (Ballister, et al., 2018); or the ability to evoke G protein-coupled inwardly-rectifying potassium channel-mediated (GIRK) currents in neurons expressing a GIRK2-1 channel, as described in details in the Examples section which follows.

According to specific embodiments, the bistable type II opsin activates Gz signaling in a cell expressing same following exposure to light in a wavelength that activates it, as determined by e.g. GsX assay (Ballister, et al., 2018).

According to specific embodiments, the bistable type II opsin is OPN3.

As used herein, the term “OPN3” refers to the vertebrate Opsin-3, also known as encephalopsin or panopsin, and any homolog thereof.

According to specific embodiments, the OPN3 is the mosquito (Anopheles stephensi) OPN3 (MosOpn3), such as provided in the following Accession Number: BAN05625.

According to specific embodiments, the MosOpn3 amino acid sequence comprises SEQ ID NO: 8.

According to specific embodiments, the MosOpn3 amino acid sequence consists of SEQ ID NO: 8.

According to specific embodiments, the MosOpn3 amino acid sequence is the amino acid sequence described in Koyanagi et al. (Proc Natl Acad Sci USA. 2013 Mar. 26; 110 (13): 4998-5003), the content of which are fully incorporated herein by reference.

According to other specific embodiments, the MosOpn3 amino acid sequence is not the amino acid sequence described in Koyanagi et al. (Proc Natl Acad Sci USA. 2013 Mar. 26; 110 (13): 4998-5003).

According to specific embodiments, the MosOpn3 amino acid sequence comprises SEQ ID NO: 9.

According to specific embodiments, the MosOpn3 amino acid sequence consists of SEQ ID NO: 9.

According to specific embodiments, the MosOpn3 amino acid sequence does not consist of SEQ ID NO: 9.

The term “MosOpn3” also encompasses functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 8; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the bistable type II opsin is TMT, also known as Teleost multiple tissue.

According to other specific embodiments, the bistable type II opsin is not TMT.

According to specific embodiments, the TMT is the pufferfish teleost multiple tissue opsin (PufTMT) such as provided in the following Accession Number: AAM90677.

According to other specific embodiments, the bistable opsin II is not the pufferfish teleost multiple tissue opsin (PufTMT).

According to specific embodiments, the PufTMT amino acid sequence comprises SEQ ID NO: 10.

According to specific embodiments, the PufTMT amino acid sequence consists of SEQ ID NO: 10.

According to specific embodiments, the TMT is TMT1A such as the medaka teleost multiple tissue opsin 1A (medakaTMT1A) such as provided in the following Accession Number: AGK24990.

According to specific embodiments, the medakaTMT1A amino acid sequence comprises SEQ ID NO: 33.

According to specific embodiments, the medakaTMT1A amino acid sequence consists of SEQ ID NO: 33.

According to specific embodiments, the PufTMT or medakaTMT1A amino acid sequence is the amino acid sequence described in Sakai K. et al. [PLOS ONE (2015) 10 (10): e0141238], the content of which are fully incorporated herein by reference.

The terms “PufTMT”, “medakaTMT1A” also encompass functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 10, 33, respectively; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the bistable type II opsin is parapinopsin. Non-limiting examples of parapinopsins include Lethenteron camtschaticum parapinopsin (LcPP), zebrafish parapinopsin 1 (zPP1), zebrafish parapinopsin 2 [zPP2, also known as Danio rerio parapinopsin2 (drPP2)], pufferfish parapinopsin (pPP2, also known as TrPP2).

According to specific embodiments, the parapinopsin is the Lethenteron camtschaticum (Lamprey) parapinopsin (LcPP) such as provided in the following Accession Number: BAD13381.

According to specific embodiments, the LcPP amino acid sequence comprises SEQ ID NO: 29.

According to specific embodiments, the LcPP amino acid sequence consists of SEQ ID NO: 29.

According to specific embodiments, the LcPP amino acid sequence is the amino acid sequence described in Eickelbeck et al. [ChemBioChem (2020) 21:612-617], the content of which are fully incorporated herein by reference.

According to specific embodiments, the parapinopsin is the zebra fish parapinopsin 1 (zPP1) such as provided in the following Accession Number: AB626966.

According to specific embodiments, the zPP1 amino acid sequence comprises SEQ ID NO: 37.

According to specific embodiments, the zPP1 amino acid sequence consists of SEQ ID NO: 37.

According to specific embodiments, the zPP1 amino acid sequence is the amino acid sequence described in Kawano-Yamashita E. et al. [PLOS ONE (2015) 10 (10): e0141280], the content of which are fully incorporated herein by reference.

According to specific embodiments, the parapinopsin is the pufferfish parapinopsin (pPP2) such as provided in the following Accession Number: AB626965.

According to specific embodiments, the pPP2 amino acid sequence comprises SEQ ID NO: 41.

According to specific embodiments, the pPP2 amino acid sequence consists of SEQ ID NO: 41.

The terms “LcPP”, “zPP1”, “pPP2” also encompass functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 29, 37, 41, respectively; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the bistable type II opsin is PdCO, also known as Platynereis dumerilii ciliary opsin.

According to specific embodiments, the PdCO is the PdCO2 such as provided in the following Accession Number: AY692353.

According to specific embodiments, the PdCO2 amino acid sequence comprises SEQ ID NO: 25.

According to specific embodiments, the PdCO2 amino acid sequence consists of SEQ ID NO: 25.

According to specific embodiments, the PdCO amino acid sequence is the amino acid sequence described in Tsukamoto et al. [J. Biol. Chem. (2017) doi: 10.1074/jbc.M117.793539], the content of which are fully incorporated herein by reference.

The term “PdCO2” also encompasses functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 25; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the bistable type II opsin is selected from the group consisting of MosOpn3, LcPP, zPP1, pPP2, PdCO2, PufTMT and medakaTMT1A.

According to specific embodiments, the bistable type II opsin is selected from the group consisting of MosOpn3, LcPP, zPP1, pPP2, PdCO2 and medakaTMT1A.

The polynucleotides disclosed herein encode a bistable type II opsin attached to an ER export signal and/or membrane trafficking signal heterologous to the bistable type II opsin.

As used herein, the term “heterologous” refers to a sequence which is not native to the bistable type II opsin at least in localization or is completely absent from the native sequence of the polypeptide. The heterologous moiety forms a chimeric or a fusion polypeptide.

According to specific embodiments, the heterologous ER export signal and/or membrane trafficking signal is located C-terminally to the bistable type II opsin.

According to specific embodiments, the heterologous ER export signal and/or membrane trafficking signal enables trafficking to axonal presynaptic terminals. To render explicit, according to specific embodiments, the ER export signal and/or membrane trafficking signal enables membrane expression or presentation of the bistable type II opsin. Methods of determining trafficking to axonal presynaptic terminals are well known in the art and include for example immunostaining and fluorescence microscopy. Alternatively or additionally, determining may be performed by electrophysiological and behavioral methods, such as disclosed for example in Mahn et al. (2021) Neuron 109:1621-1635.

ER export signals are known in the art, and disclosed e.g. in Stockklausner et al., FEBS Lett.; 493 (2-3): 129-133 March 2001; Ma et al., Science Vol. 291. no. 5502:316-319, 2001); Paulhe et al., J. Biol. Chem., Vol. 279, Issue 53, 55545-55555, Dec. 31, 2004); Farhan et al., J. Cell Sci. 121:753-761, Feb. 19, 2008; the contents of each are incorporated herein by reference in their entirety.

According to specific embodiments, the ER export signal is of a protein expressed in neuronal cells.

According to specific embodiments, the ER export signal is of a protein expressed in the axons or the presynaptic terminals of neuronal cells.

Non-limiting examples ER export signals can be the signals of the inward rectifier potassium channel Kir2.1, NgCAM, VAMP2, Neurexin, Synapsin, Synaptophysin, Synaptotagmin, SynCAM, Piccolo or Basoon.

According to specific embodiments, the ER signal is of the inward rectifier potassium channel Kir2.1.

Non-limiting examples of amino acid sequence of ER export signals that can be used with specific embodiments of the invention include, FXYENE (SEQ ID NO: 11, where X is any amino acid), e.g. FCYENEV (SEQ ID NO: 2); VXXSL (where X is any amino acid), e.g. VKESL (SEQ ID NO: 13); VLGSL (SEQ ID NO: 14); NANSFCYENEVALTSK (SEQ ID NO: 15); C-terminal valine residue; and VMI.

According to specific embodiments, the amino acid sequence of the ER export signal is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to an amino acid sequence selected from the group consisting of SEQ ID NO: 2, 13 and 14, each possibility represents a separate embodiments of the invention.

According to specific embodiments, the amino acid sequence of the ER export signal comprises SEQ ID NO: 2.

According to specific embodiments, the amino acid sequence of the ER export signal consists of SEQ ID NO: 2.

According to specific embodiments, the ER export signal amino acid sequence is 5-25 amino acids in length, e.g. 5-10, 10-15, 15-20, 20-25 amino acids in length.

Membrane trafficking signals are known in the art, and include, but are not limited to membrane trafficking signals of a protein expressed on the membranes of neuronal cells.

According to specific embodiments, the membrane trafficking signal is of a protein expressed in neuronal cells.

According to specific embodiments, the membrane trafficking signal is of a protein expressed in the axons or the presynaptic terminals of neuronal cells.

Non-limiting examples of membrane trafficking signals can be the signals of the inward rectifier potassium channel Kir2.1, the hChR2, the neuronal nicotinic acetylcholine receptor, NgCAM, VAMP2, Neurexin, Synapsin, Synaptophysin, Synaptotagmin, SynCAM, Piccolo or Basoon.

According to specific embodiments, the trafficking signal is of a Kir2.1 polypeptide.

Amino acid sequence of trafficking sequences that are suitable for use with specific embodiments include, but are not limited to KSRITSEGEYIPLDQIDINV (SEQ ID NO: 1), MDYGGALSAVGRELLFVTNPVVVNGS ID NO: (SEQ 16), MAGHSNSMALFSFSLLWLCSGVLGTEF (SEQ ID NO 17), MGLRALMLWLLAAAGLVRESLQG (SEQ ID NO: 18), MRGTPLLLVVSLFSLLQD (SEQ ID NO: 19).

According to specific embodiments, the amino acid sequence of membrane trafficking signal is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to an amino acid sequence selected from the group consisting of SEQ ID NO: 1, 16, 17, 18 and 19, each possibility represents a separate embodiments of the invention.

According to specific embodiments, the amino acid sequence of the membrane trafficking signal comprises SEQ ID NO: 1.

According to specific embodiments, the amino acid sequence of the membrane trafficking signal consisting of SEQ ID NO: 1.

According to specific embodiments, the membrane trafficking signal amino acid sequence is 10-50 amino acids in length, e.g. 10-20, 20-30, 30-40, 40-50 amino acids in length.

As used herein the term “polynucleotide”, refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

According to specific embodiments, any of the polynucleotides and nucleic acid sequences disclosed herein may comprise conservative nucleic acid substitutions. Conservatively modified polynucleotides refer to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations”, which are one species of conservatively modified polynucleotides. According to specific embodiments, any polynucleotide and nucleic acid sequence described herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a polynucleotide which encodes a polypeptide is implicit in a described sequence with respect to the expression product.

According to specific embodiments, the polynucleotides or nucleic acid sequences disclosed herein are codon optimized to heterologous (e.g. mammalian) expression.

Methods of codon optimization are known in the art and disclosed e.g. in Grote et al. (Nucleic Acid Res. Nucleic Acids Res. (2005) July 1; 33 (Web Server issue): W526-W531) and include e.g. mouse codon usage optimized or human codon usage optimized versions.

Hence, according to specific embodiments, the nucleic acid sequence of the MosOpn3 comprises SEQ ID NO: 20.

According to specific embodiments, the nucleic acid sequence of the MosOpn3 consists of SEQ ID NO: 20.

According to specific embodiments, the nucleic acid sequence of the MosOpn3 comprises SEQ ID NO: 21.

According to specific embodiments, the nucleic acid sequence of the MosOpn3 consists of SEQ ID NO: 21.

According to specific embodiments, the nucleic acid sequence of the PufTMT comprises SEQ ID NO: 22.

According to specific embodiments, the nucleic acid sequence of the PufTMT consists of SEQ ID NO: 22.

According to specific embodiments, the nucleic acid sequence of the PufTMT comprises SEQ ID NO: 23.

According to specific embodiments, the nucleic acid sequence of the PufTMT consists of SEQ ID NO: 23.

According to specific embodiments, the nucleic acid sequence of the medakaTMT1A comprises SEQ ID NO: 34.

According to specific embodiments, the nucleic acid sequence of the medakaTMT1A consists of SEQ ID NO: 34.

According to specific embodiments, the nucleic acid sequence of the LcPP comprises SEQ ID NO: 30.

According to specific embodiments, the nucleic acid sequence of the LcPP consists of SEQ ID NO: 30.

According to specific embodiments, the nucleic acid sequence of the zPP1 comprises SEQ ID NO: 38.

According to specific embodiments, the nucleic acid sequence of the zPP1 consists of SEQ ID NO: 38.

According to specific embodiments, the nucleic acid sequence of the pPP2 comprises SEQ ID NO: 42.

According to specific embodiments, the nucleic acid sequence of the pPP2 consists of SEQ ID NO: 42.

According to specific embodiments, the nucleic acid sequence of the PdCO2 comprises SEQ ID NO: 26.

According to specific embodiments, the nucleic acid sequence of the PdCO2 consists of SEQ ID NO: 26.

To express an exogenous polypeptide in mammalian cells, a polynucleotide encoding the polypeptide is preferably ligated into a nucleic acid construct suitable for mammalian cell expression. Such a nucleic acid construct includes a promoter sequence for directing transcription of the polynucleotide sequence in the cell in a constitutive or inducible manner.

Hence, according to specific embodiments, the polynucleotide is comprised a in a nucleic acid construct comprising the polynucleotide and a regulatory element for directing expression of the polynucleotide in a cell (e.g. promoter).

According to specific embodiments, the regulatory element is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Thus, according to specific embodiments, promoter is a neuron specific promoter. Non-limiting examples of neuron-specific promoters include the neurofilament promoter [Byrne et al. (1989) Proc. Natl. Acad. Sci. USA 86:5473-5477; or GenBank HUMNFL, L04147], neuron-specific enolase (NSE) promoter (see, e.g., EMBL HSENO2, X51956; see also, e.g., U.S. Pat. Nos. 6,649,811, 5,387,742); aromatic amino acid decarboxylase (AADC) promoter; synapsin promoter (see, e.g., GenBank HUMSYNIB, M55301); thy-1 promoter (see, e.g., Chen et al. (1987) Cell 51:7-19; and Llewellyn et al. (2010) Nat. Med. 16:1161); serotonin receptor promoter (see, e.g., GenBank S62283); tyrosine hydroxylase promoter (TH) (see, e.g., Nucl. Acids. Res. 15:2363-2384 (1987) and Neuron 6:583-594 (1991)); GnRH promoter (see, e.g., Radovick et al., Proc. Natl. Acad. Sci. USA 88:3402-3406 (1991)); L7 promoter (see, e.g., Oberdick et al., Science 248:223-226 (1990)); DNMT promoter (see, e.g., Bartge et al., Proc. Natl. Acad. Sci. USA 85:3648-3652 (1988)); enkephalin promoter (see, e.g., Comb et al., EMBO J. 17:3793-3805 (1988)); a myelin basic protein (MBP) promoter; CMV enhancer/platelet-derived growth factor-β promoter (see, e.g., Liu et al. (2004) Gene Therapy 11:52-60); motor neuron-specific gene Hb9 promoter (see, e.g., U.S. Pat. No. 7,632,679; and Lee et al. (2004) Development 131:3295-3306); and alpha subunit of Ca⁽²⁺⁾-calmodulin-dependent protein kinase II (CaMKIIa) promoter (see, e.g., Mayford et al. (1996) Proc. Natl. Acad. Sci. USA 93:13250).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long terminal repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference. Enhancers specific for distinct neuronal cell types that can be included in AAV expression vectors to gain specificity without a Cre-driver line have also been described in the arts and described e.g. in Hrvatin et al. (doi:www://doi(dot)org/10.1101/570895), which is incorporated herein by reference. Cell-type specific enhancers, such as described in e.g. Jüttner e al. [Nature Neuroscience volume 22, pages 1345-1356 (2019)] or Dimidschstein et al. (Nature Neuroscience volume 19, pages 1743-1749 (2016)], the contents of which are incorporated herein by reference, for expression in inhibitory interneurons.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the polynucleotide.

Thus, according to specific embodiments, the polynucleotide or vector comprises a genomic integration sequence, such that upon administration, the polynucleotide is integrated in the genome of the infected or transfected cell.

According to other specific embodiments, the polynucleotide or vector does not comprise a genomic integration sequence, such that upon administration the polynucleotide does not integrate with the genome of the infected or transfected cell. In such cases, specific embodiments suggest the formation of an episome.

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail”configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1 (+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine 15 mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein.

Recombinant viral vectors are useful for in vivo expression of the polypeptides since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Any of the components comprised in the polynucleotide as described herein may be linked to each other directly or via a linker, each possibility represents a separate embodiment of the present invention.

Any linker known in the art can be used with specific embodiments of the invention.

According to specific embodiments, the linker may be derived from naturally-occurring multi-domain proteins or is an empirical linker as described, for example, in Chichili et al., (2013), Protein Sci. 22 (2): 153-167, Chen et al, (2013), Adv Drug Deliv Rev. 65 (10): 1357-1369, the entire contents of which are hereby incorporated by reference. In some embodiments, the linker may be designed using linker designing databases and computer programs such as those described in Chen et al., (2013), Adv Drug Deliv Rev. 65 (10): 1357-1369 and Crasto et al., (2000), Protein Eng. 13 (5): 309-312, the entire contents of which are hereby incorporated by reference.

According to specific embodiments, the amino acid sequence of the linker is selected from the group consisting of PRARDP (SEQ ID NO: 4), (Gly), (where n indicates variable copy numbers), (GₙSₙ)ₙ (where n indicates variable copy numbers), ((GₙSₙ)ₙPₙ)ₙ (where n indicates variable copy numbers) and (EAAAK)ₙ (where n indicates variable copy numbers, SEQ ID NO: 24).

According to specific embodiments, the polynucleotide may comprise or encode epitope tags, fluorescent proteins, cleavable linker peptides, a cell penetrating moiety, targeting moieties and the like.

According to specific embodiments, the polynucleotide encodes an amino acid sequence for directing the bistable type II opsin to a specific membrane location e.g. the axon or the presynaptic terminal.

Various methods can be used to introduce the polynucleotide or expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. Introduction of nucleic acids by viral infection offers several advantages over other methods such as lipofection and electroporation, since higher transfection efficiency can be obtained due to the infectious nature of viruses. Thus, according to specific embodiments, the polynucleotide is packed in a viral vector (e.g. AAV). Other vectors can be used that are non-viral, such as cationic lipids, polylysine, and dendrimers.

The polynucleotides and nucleic acid constructs of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polynucleotides, nucleic acid constructs and polypeptides encoded therefrom accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen. Thus, for example, for injection, the active ingredients of the pharmaceutical composition may be formulated in aqueous solutions, preferably in physiologically compatible buffers such as Hank's solution, Ringer's solution, or physiological salt buffer. Alternatively, the active ingredient may be in powder form for constitution with a suitable vehicle, e.g., sterile, pyrogen-free water based solution, before use.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

Non-limiting examples of animal models that can be used with specific embodiments of the invention are described in the Examples section which follows and include for example the genetic Slc6a1 model or the SCN1a (Dravet) genetic model for generalized onset epilepsy and the Kainate model for focal onset epilepsy and (see www(dot)informatics.jax(dot)org/marker/MGI: 95627; Lindquist et al. bioRxiv preprint doi: www(dot)doi(dot)org/10.1101/2021.12.17.473036; Lévesque M, et al. (20123) Neurosci Biobehav Rev. 37(10 Pt 2): 2887-99; and Twele F et al. (2017) Epilepsia Open. 2 (2): 180-187). Closed- and open-loop approaches are described for example in Paz et al. Nat Neurosci (2013)16(1): 64-70; and Salanova V. et al.; SANTÉ Study Group. (2021) Epilepsia. 62(6): 1306-1317, the contents of which are fully incorporated herein by reference.

The doses determined in the rodent animal model can be converted for the treatment of other species such as human and other animals diagnosed with the disease, using conversion Tables known to those skilled in the art.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration.

Depending on the severity and responsiveness of the condition to be treated, dosing can be of a single or a plurality of administrations, with course of treatment lasting from several days to several weeks or until cure is effected or diminution of the disease state is achieved, as further described hereinbelow.

The amount of a composition to be administered will, of course, be dependent on the subject being treated, the severity of the affliction, the manner of administration, the judgment of the prescribing physician or neurologist, etc. According to specific embodiments, the amount to be administered depends on the judgment of the neurosurgeon, in accordance with electrophysiological measurements (e.g. EEG) or imaging e.g. MRI.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Following administration of the polynucleotide, the methods disclosed herein comprise exposing a neural region of the subject to light in a wavelength that activates the bistable type II opsin.

Such a wavelength typically depends on the type of the bistable type II opsin. Determining the suitable wavelength is well within the capabilities of the skilled in the art. According to specific embodiments, the light may range from ultraviolet down to near-infrared light. According to specific embodiments, the light is an ultraviolet, blue, green, yellow or red light.

According to specific embodiments, the wavelength is 350-670 nm.

According to specific embodiments, the wavelength is 450-650 nm.

According to specific embodiments, the wavelength is 450-490 nm.

According to specific embodiments, the wavelength is about 470 nm.

According to specific embodiments, the wavelength is 540-580 nm.

According to specific embodiments, the wavelength is about 560 nm.

According to specific embodiments, the wavelength is 610-650 nm.

According to specific embodiments, the wavelength is about 630 nm.

According to specific embodiments, exposing to light is effected by light pulses that can have a duration for any of at least 1 millisecond (ms), at least 5 ms, at least 10 ms, at least 50 ms, at least 100 ms, at least 500 ms, at least 1 sec, at least 5 sec, at least 10 sec, at least 20 sec, at least 30 sec, at least 40 sec.

According to specific embodiments, exposing to light is effected by light pulses that can have a duration for any of about 1 millisecond (ms), about 2 ms, about 3, ms, about 4, ms, about 5 ms, about 6 ms, about 7 ms, about 8 ms, about 9 ms, about 10 ms, about 15 ms, about 20 ms, about 25 ms, about 30 ms, about 35 ms, about 40 ms, about 45 ms, about 50 ms, about 60 ms, about 70 ms, about 80 ms, about 90 ms, about 100 ms, about 200 ms, about 300 ms, about 400 ms, about 500 ms, about 600 ms, about 700 ms, about 800 ms, about 900 ms, about 1 sec, about 1.25 sec, about 1.5 sec, about 2 sec, about 5 sec, about 10 sec, about 20 sec, about 30 sec, about 40 sec.

According to specific embodiments, exposing to light is effected by a light pulse having a duration of 0.5-30 seconds.

According to specific embodiments, the neural region comprises a cell body and/or an axon of the thalamus nucleus. Such neural regions typically include the thalamus and the cortex.

According to specific embodiments, the neural region comprises an axon terminal of the thalamus nucleus.

According to specific embodiments, the neural region comprises a presynaptic terminal of an axon of the thalamus nucleus.

Thalamic-cortical projections are well known in the art and documented for example in Zheng et al. (2023) Expert Rev Neurother. 23(2): 123-140; Halassa and Sherman (2019) Neuron 103(5): P762-770; Zhang et al. (2015) Neuroimage Clin. 9:117-127.; and Law et al. (2018) AJNR Am J Neuroradiol. 39 (8): 1523-1529. Hence, determination of such a neural region is well within the capabilities of those skilled in the art, and depends on the specific thalamus nucleus injected. Thus, for example, upon administration to the anterior nucleus of thalamus (ANT) exposing may be effected at frontal cortical areas, anterior cingulate cortex, retrosplenial cortex and/or subicular cortex. This modality may be used to treat e.g. temporal lobe epilepsy (which originates from the hippocampus). Another non-limiting example, upon administration to the Centromedian nucleus exposing may be effected at the cortex and basal ganglia. This modality may be used to treat e.g. generalized onset epilepsies (like LGS, Slc6a1, Dravet and others).

Methods and apparatuses for exposing such neural regions to such wavelengths are known in the art and include for example the Clinatec device for PhotobioModulation therapy disclosed in www(dot)nature(dot)com/articles/d41586-023-00079-0 and the Blackrock Neurotech's Opto-array described in www(dot)nature(dot)com/articles/s41592-021-01238-9e. According to specific embodiments, exposing is effected using a skull-mounted device. For example, the device may comprise an uLED array aimed to be placed extracranially, and is conjoined with epidural, subdural or depth electrodes aimed at recording electrophysiological neural activity from various brain locations. According to specific embodiments, the device allows electrical recording and transcranial optical stimulation in parallel—in closed-loop and/or open-loop manners.

Care should be taken to expose the neural regions to light following an amount of time allowing expression of the polypeptide encoded by the polynucleotide or the nucleic acid construct. Hence, according to specific embodiments, exposing is effected following an amount of time allowing expression of the bistable type II opsin in a neural cell of the thalamus nucleus and its axonal presynaptic terminals. According to specific embodiments, exposing is effected following an amount of time allowing membrane expression or presentation of the bistable type II opsin. Thus, according to specific embodiments, the exposing is effected at least 1 week, at least 2 weeks, at least 3, weeks, at least 4 weeks, at least 5 weeks, at least 6 weeks, at least 7 weeks, at least 8 weeks, at least 9 weeks, at least 10 weeks following the administering.

According to a specific embodiment, exposing is effected at least 6 weeks following said administering.

According to a specific embodiment, exposing is effected at least 8 weeks following said administering.

Exposing to light may be effected independent or dependent of detection of an epileptic seizure.

According to specific embodiments, exposing to light is effected upon detection of an epileptic seizure (may be also referred to as the “closed-loop approach”). Such detection may be for example by electrical means, e.g. by EEG, ECOG, S-EEG and the like (according to amplitude, spiking, wave pattern) or by detection of symptoms e.g. tremor, stiffness, or any other symptom such as disclosed herein above; and can be effected automatically by a device or by the subject or caretaker.

Thus, for example, the skull-mounted device may allow electrophysiological (e.g. EEG, EGOG, S-EEG) recording; such that upon detection of an epileptic seizure (e.g. using an algorithm identifying initiation of an epileptic seizure sequence) a light pulse(s) will be transmitted.

Alternatively, of additionally, the subject may actively start illumination upon detecting symptoms suspected to allude at the imminent onset of a seizure (e.g. see the description on aura hereinabove).

According to other specific embodiments, exposing to light comprises repeated illumination independent (may be also referred to as the “open-loop approach”) of detection of an epileptic seizure.

Under such a scenario, illumination is delivered in a preset cycle and not directly in response to an epileptic seizure and may be either in a continuous (or chronic) or alternating manner.

As the bistable type II opsins of some embodiments of the invention remain active for about 5 minutes following an illumination pulse; a light pulse every 10 seconds-5 minutes will result in a continuous active opsin and a light pulse every more than 5 minutes (e.g. every 10-30 minutes) will result in alternating activation of the opsin.

The preset cycle may be controlled by a pre-set program and/or by the subject in an active manner (e.g. before performing an activity such as driving and the like).

It should be noted that according to specific embodiments, it is possible to reverse activation of the expressed bistable II opsin using light in a wavelength different than the one that activates it, enabling easier regulation of the amount and duration of activation. Thus, according to specific embodiments, the method comprising exposing the neural region of the subject comprising the cell body and/or axon of the thalamus nucleus, to light in a wavelength that inhibits activation of the polypeptide.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques.

### Example 1

eOPN3 Thalamic Targeted Seizure Inhibition

A mosquito (Anopheles stephensi)-derived homolog of the human encephalopsin protein (OPN3), a bistable type II opsin (i.e. remain bound to the retinal chromophore after illumination and display prolonged signal transduction following a single illumination pulse) that can be expressed on membranes of rat hippocampal neurons and most importantly in distal axonal presynaptic terminals by the addition of an ER export signal and membrane trafficking signal of a Kir2.1 protein was previously described International Patent Application Publication No. WO2020/188572, and referred to herein as “eOPN3”.

In an effort to control epileptic seizures through specific inhibition of thalamic projections to the cortex (the thalamo-cortical circuit), eOPN3 is administered to thalamic nuclei of epileptic animal models followed by illumination of cell bodies and/or their terminals.

Two different animal models are used:
Slc6a1 mutated rodent model, a genetic generalized type epilepsy model (see www(dot)informatics.jax(dot)org/marker/MGI: 95627 and Lindquist et al. bioRxiv preprint doi:www(dot)doi(dot)org/10.1101/2021.12.17.473036); and
Kainate mutated rodent model, a focal-onset epilepsy model (see Lévesque M, et al. (20123) Neurosci Biobehav Rev. 37(10 Pt 2): 2887-99; and Twele F et al. (2017) Epilepsia Open. 2(2): 180-187).

Recombinant AAV vectors encoding eOPN3 are produced as described in International Patent Application Publication No. WO2020/188572. Briefly, a construct encoding the OPN3 opsin is subcloned into pAAV vectors under the CamKIIα promoter and in-frame with mScarlet at the C-terminus. A nucleic acid encoding the Kir2.1 membrane trafficking signal (KSRITSEGEYIPLDQIDINV, SEQ ID NO: 1) was added between the opsin and the mScarlet coding sequences and a nucleic acid encoding the Kir2.1 ER export signal (FCYENEV, SEQ ID NO: 2) was added following the C-terminus of mScarlet. The sequences of the eOPN3-mScarlet open reading frames are provided in SEQ ID NOs: 5-6 (the mScarlet nucleic acid sequence is provided in SEQ ID NO: 7).

Rodents carrying the respective condition (Slc6a1/Kainate) are stereotactically injected bilaterally into the thalamus with the viral vector. Control animals are similarly injected with a control agent (AAV containing GFP fluorophore transgene). Mice are allowed to recover for 6-8 weeks to allow for viral expression and intracellular transmission of the opsin to the terminals in the cortex.

Following the aforementioned waiting period, the animals are implanted with a skull-mounted device that allows electrophysiological recording as well as illumination. The efficacy of illuminating eOPN3-expressing thalamic terminals in the cortex on seizure control (amount, extent and/or duration) is then measured using either an open (continuous illumination for a set period of time) or a closed-loop (autonomous illumination operated by a seizure detection algorithm software) illumination protocols. The results are compared to no illumination periods.

Endpoints measures include seizure frequency reduction, seizure duration reduction, seizure symptoms reduction, expression patterns, etc.

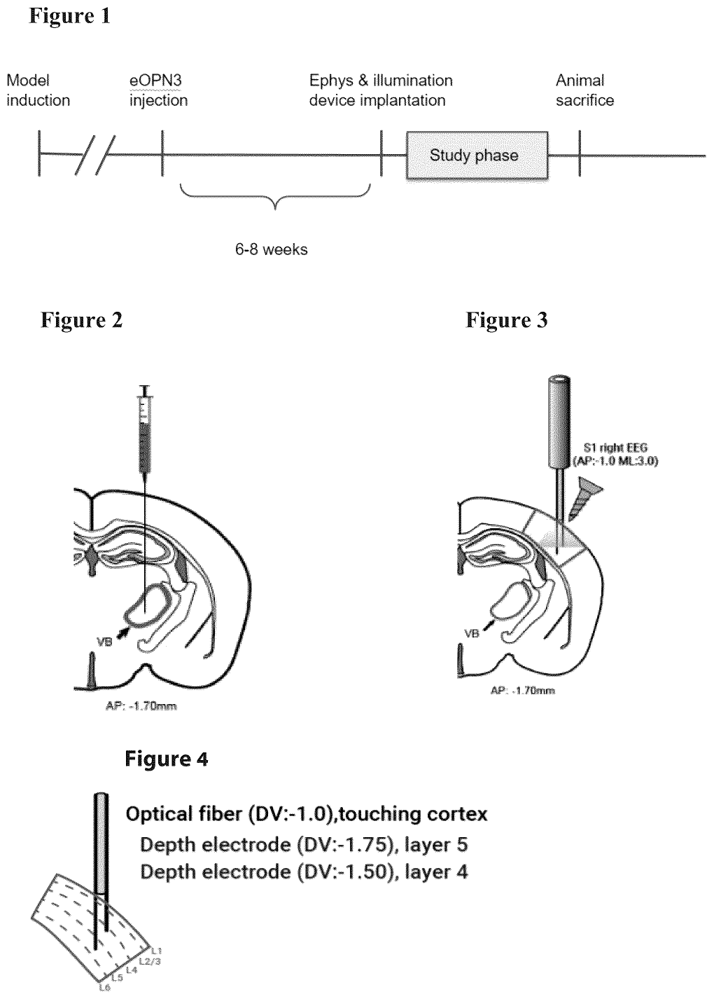

The experimental timeline and a summing outline table are provided in FIG. 1 and Table 1 hereinbelow.

TABLE 1
Outline table

| | | Site of | Site of | |
|---|---|---|---|---|
| Indication | Agent | administration | expression | Site of illumination |
| Focal onset epilepsy (e.g. | AVV | Thalamic | Thalamic | The injected thalamus |
| Temporal Lobe epilepsy, | encoding | nuclei (e.g. | neurons - | nuclei |
| frontal lobe epilepsy, | eOPN3 | ANT, CM, VB, | cell bodies | or regions within the |
| Occipital Lobe Epilepsy, | | VM, CL, | and axon | thalamus, cortex or other |
| Epilepsia Partialis | | Pulvinar, etc.) | terminals | regions having |
| Continua (EPC), etc.) | | | | projections with the |
| Generalized epilepsy (e.g. | | | | respective administration |
| Developmental | | | | site |
| Epilepsies & | | | | |
| Encephalopathies | | | | |
| (DEEs), LGS, Dravet, | | | | |
| Slc6a1, WWOX, | | | | |
| Absence,, Rasmussen's | | | | |
| syndrome, etc.) | | | | |

### Example 2

eOPN3 Thalamic Targeted Seizure Inhibition

### Study Design:

1. Injection of 500 nL of eOPN3 encoding AAVs, such as AAV9-CaMKIIa-eOPN3-mScarlet (produced as described in Example 1 hereinabove) unilaterally into thalamic nuclei such as—ANT, CM, VB, CL, Pulvinar or others, of wild-type B6 mice (FIG. 2);
2. 4 weeks following the injection implantation of devices that contain an optical fiber with depth electrodes in the ipsilateral S1 cortex+EEG screws (FIG. 3). A different version of the illumination device can include LEDs or μLEDs for illumination with or without depth electrodes.
3. 6-8 weeks following the injection seizures are generated (under anesthesia) using substances such as Pentylenetetrazole (PTZ), Gamma ButyroLactone (GBL), Picrotoxin (PTX), Kainic Acid (KA) etc.
4. Illumination of cortical projections terminals using illumination devices (detailed above) in response to the identification of seizures, or unrelated to seizure detection (open-loop) in order to prevent seizure activity.
5. Simultaneous recording of electrical activity using depth and cortical electrodes (a schematic representation is provided in FIGS. 3-4) in order to detect the seizures and obtain endpoints data.
6. Endpoints to be measured include seizure frequency reduction, seizure duration reduction, Stage 5 (Racine score) seizures reduction, etc.

### Results

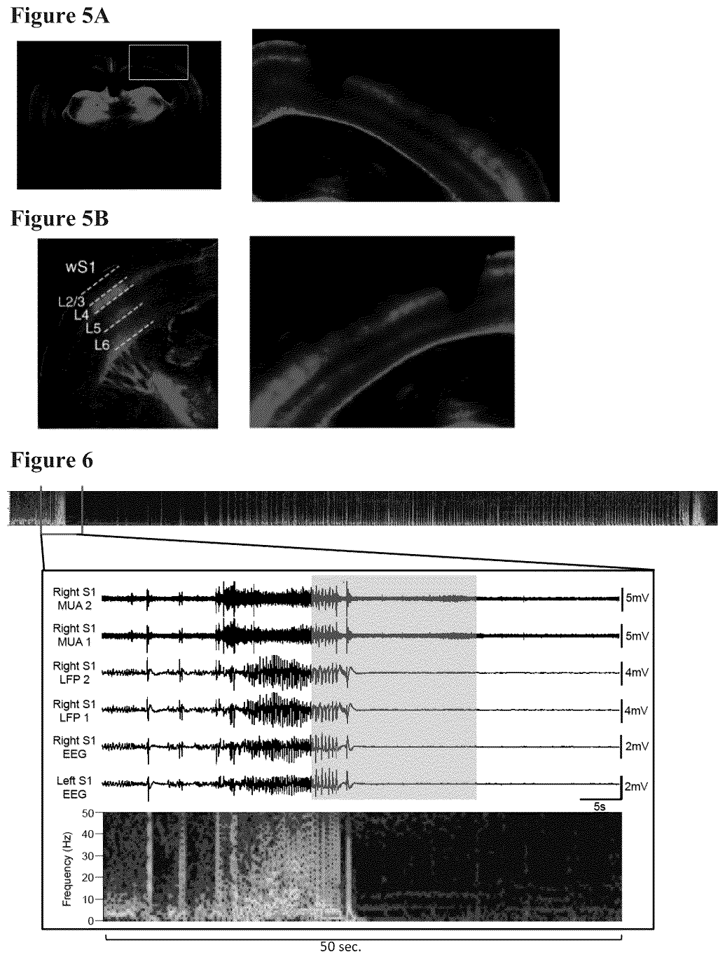

FIGS. 5A-B demonstrate that injection of recombinant AAV vectors encoding eOPN3 into the thalamus of mice lead to robust expression in the neuron bodies (somas) in the thalamus, as well as in projections in the cortex (axons) seen in several cortical layers (mostly Layers 4 and 5).

Furthermore, using the generalized seizure mouse model initiated by PTZ, illumination of cortical projections of VB thalamic neurons expressing eOPN3 halted seizure activity (FIG. 6).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating epilepsy in a subject in need thereof, the method comprising:
(a) administering into a thalamus nucleus of the subject a therapeutically effective amount of a polynucleotide encoding a bistable type II opsin attached to a heterologous ER export signal and/or membrane trafficking signal which enables trafficking of said bistable type II opsin to axonal presynaptic terminals; and
(b) exposing a neural region of said subject to light in a wavelength that activates said bistable type II opsin, wherein said neural region comprises a cell body and/or an axon of said thalamus nucleus,
thereby treating epilepsy in the subject.

2. A method of treating epilepsy in a subject in need thereof having been administered into a thalamus nucleus a therapeutically effective amount of a polynucleotide encoding a bistable type II opsin attached to a heterologous ER export signal and/or membrane trafficking signal which enables trafficking of said bistable type II opsin to axonal presynaptic terminals, the method comprising exposing a neural region of said subject to light in a wavelength that activates said bistable type II opsin, wherein said neural region comprises a cell body and/or an axon of said thalamus nucleus, thereby treating epilepsy in the subject.

3. The method of claim 1, wherein said epilepsy is a generalized onset epilepsy.

4. The method of claim 3, wherein said generalized onset epilepsy is selected from the group consisting of Developmental Epilepsy & Encephalopathy (DEE), drug-resistant generalized epilepsy, Lennox-Gastaut Syndrome (LGS), Dravet syndrome, epilepsy associated with a mutation in Slc6a1, epilepsy associated with a mutation in WWOX, generalized epilepsy of unknown origin, multifocal epilepsy, Absence epilepsy and Rasmussen's syndrome.

5. The method of claim 1, wherein said epilepsy is a focal onset epilepsy.

6. The method of claim 5, wherein said focal onset epilepsy is selected from the group consisting of drug-resistant focal epilepsy, temporal lobe epilepsy, frontal lobe epilepsy, parietal lobe epilepsy, Sturge Weber syndrome, Tuberous Sclerosis Complex, post-stroke epilepsy, post TBI epilepsy, focal cortical dysplasia, occipital lobe epilepsy and Epilpesia Partialis Continua (EPC).

7. The method of claim 5, wherein said focal onset epilepsy comprises secondary generalized seizures.

8. The method of claim 1, wherein said administering or said administration is by a stereotactic injection.

9. The method of claim 1, wherein said thalamus nucleus is selected from the group consisting of Anterior Nucleus of the Thalamus (ANT), Intralaminar nucleus, lateral thalamic nucleus, medial thalamic nucleus, Centromedian Nucleus (CM), Ventral Posterior Nucleus (VB), Ventral Medial Nucleus (VM), Central Lateral (CL) and Pulvinar.

10. The method of claim 1, wherein said polynucleotide is packed in a viral vector.

11. The method of claim 1, wherein said bistable type II opsin is selected from the group consisting of OPN3, OPN4, OPN5, LcPP, DrPP2, TrPP2, parapinopsin, PdCO, TMT and peropsin.

12. The method of claim 1, wherein said bistable type II opsin is OPN3; and optionally wherein said OPN3 is mosquito OPN3 (MosOpn3).

13. The method of claim 1, wherein said ER export signal and/or said membrane trafficking signal is of a protein expressed in neuronal cells; and optionally wherein said protein is Kir2.1.

14. The method of claim 1, wherein an amino acid sequence of said ER export signal comprises SEQ ID NO: 2; and/or wherein an amino acid sequence of said membrane trafficking signal comprises SEQ ID NO: 1.

15. The method of claim 1, wherein said exposing is effected at least 6 or 8 weeks following said administering.

16. The method of claim 1, wherein said exposing comprises repeated illumination independent of detection of an epileptic seizure.

17. The method of claim 1, wherein said exposing is effected upon detection of an epileptic seizure.

18. The method of claim 1, wherein said neural region comprises a thalamus region.

19. The method of claim 1, wherein said neural region comprises a cortex region.

20. The method of claim 1, wherein said neural region comprises a presynaptic terminal of said axon.


SEQUENCE LISTING (44 sequences)

SEQ ID NO: 1 - protein, 20 aa, synthetic construct
  REGION 1..20: note = Membrane trafficking signal
KSRITSEGEYIPLDQIDINV

SEQ ID NO: 2 - protein, 7 aa, synthetic construct
  REGION 1..7: note = ER EXPORT SIGNAL
FCYENEV

SEQ ID NO: 3 - skipped

SEQ ID NO: 4 - protein, 6 aa, synthetic construct
  REGION 1..6: note = Linker sequence
PRARDP

SEQ ID NO: 5 - protein, 607 aa, synthetic construct
  REGION 1..607: note = eOPN3-mScarlet open reading frame AA sequence
MYDAPNDVASSVADYEDLMAPWAYNAAAITLFFIGFFGFFLNLFVIALMSKDMQLWTPMNIILFNLVCSDFSVSIIGNPLTLTSAISHRWIFGRTLCVAYGFFMSLLGITSITTLTVLSYERYCLISRPFSSRNLSRKGAFLAIFFIWGYSFALTSPPLFGWGAYVQEAANISCSVNWESQTKNATTYIIFLFVFGLVVPLIVIVYSYTNIIVYMRRNSARVGRINRAEQRVTSMVAVMIVAFMVAWTPYAIFALIEQFGPPELIGPGLAVLPALIAKSSICYNPIIYVGMNTQFRAAFTRVRNKGGVPTADQNTTTMQRELTKSSRDMVTETSQVAPAPRARDPTGKSRITSEGEYIPLDQIDINVVMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKFCYENEV

SEQ ID NO: 6 - DNA, 1824 bp, synthetic construct
  misc_feature 1..1824: note = eOPN3 nucleic acid sequence
  source 1..1824: mol_type = other DNA
atgtacgacgcccccaacgacgtggccagcagcgtggccgactacgaggacctgatggccccctgggcctacaacgccgccgccatcaccctgttcttcatcggcttcttcggcttcttcctgaacctgttcgtgatcgccctgatgagcaaggacatgcagctgtggacccccatgaacatcatcctgttcaacctggtgtgcagcgacttcagcgtgagcatcatcggcaaccccctgaccctgaccagcgccatcagccacaggtggatcttcggcaggaccctgtgcgtggcctacggcttcttcatgagcctgctgggcatcaccagcatcaccaccctgaccgtgctgagctacgagaggtactgcctgatcagcaggcccttcagcagcaggaacctgagcaggaagggcgccttcctggccatcttcttcatctggggctacagcttcgccctgaccagcccccccctgttcggctggggcgcctacgtgcaggaggccgccaacatcagctgcagcgtgaactgggagagccagaccaagaacgccaccacctacatcatcttcctgttcgtgttcggcctggtggtgcccctgatcgtgatcgtgtacagctacaccaacatcatcgtgtacatgaggaggaacagcgccagggtgggcaggatcaacagggccgagcagagggtgaccagcatggtggccgtgatgatcgtggccttcatggtggcctggaccccctacgccatcttcgccctgatcgagcagttcggcccccccgagctgatcggccctggactggctgtgctgcctgctctgatcgccaagagcagcatctgctacaaccccatcatctacgtgggcatgaacacccagttcagggccgccttcaccagggtgaggaacaagggcggcgtgcccaccgccgaccagaacaccaccaccatgcagagggagctgaccaagagcagcagggacatggtgaccgagaccagccaggtggctcctgctccgcgagcccgagatccaaccggtaagagcaggatcaccagcgagggcgagtacatccccctggaccagatcgacatcaacgtggtgatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagttctgctacgagaacgaggtgtaa

SEQ ID NO: 7 - DNA, 696 bp, synthetic construct
  misc_feature 1..696: note = mScarlet NA sequence
  source 1..696: mol_type = other DNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 8 - protein, 429 aa, synthetic construct
  REGION 1..429: note = mosopn3 aa sequence
MYDAPNDVASSVADYEDLMAPWAYNAAAITLFFIGFFGFFLNLFVIALMSKDMQLWTPMNIILFNLVCSDFSVSIIGNPLTLTSAISHRWIFGRTLCVAYGFFMSLLGITSITTLTVLSYERYCLISRPFSSRNLSRKGAFLAIFFIWGYSFALTSPPLFGWGAYVQEAANISCSVNWESQTKNATTYIIFLFVFGLVVPLIVIVYSYTNIIVYMRRNSARVGRINRAEQRVTSMVAVMIVAFMVAWTPYAIFALIEQFGPPELIGPGLAVLPALIAKSSICYNPIIYVGMNTQFRAAFTRVRNKGGVPTADQNTTTMQRELTKSSRDMVECSFDFCRKKNRFKISLVKPTAPLAVVDVSSSSHPGKVTSRSPLDQTVLNEMNDEERGRERSGAGYAGSRFVRPDFELSVINSGKSILIKSKNFRSNLL

SEQ ID NO: 9 - protein, 330 aa, synthetic construct
  REGION 1..330: note = mosopn3 mutant without the last 99 aa
MYDAPNDVASSVADYEDLMAPWAYNAAAITLFFIGFFGFFLNLFVIALMSKDMQLWTPMNIILFNLVCSDFSVSIIGNPLTLTSAISHRWIFGRTLCVAYGFFMSLLGITSITTLTVLSYERYCLISRPFSSRNLSRKGAFLAIFFIWGYSFALTSPPLFGWGAYVQEAANISCSVNWESQTKNATTYIIFLFVFGLVVPLIVIVYSYTNIIVYMRRNSARVGRINRAEQRVTSMVAVMIVAFMVAWTPYAIFALIEQFGPPELIGPGLAVLPALIAKSSICYNPIIYVGMNTQFRAAFTRVRNKGGVPTADQNTTTMQRELTKSSRDMV

SEQ ID NO: 10 - protein, 288 aa, synthetic construct
  REGION 1..288: note = PufTMT amino acid sequence
MSRTGHTVVAVMLGTILLAGVFGNSVVFLVFVKYRSLRTPINLILLNISLSDILVCVFGTPLSFAASLKGRWLLGERGCEWYGFANSLFGIVSLVSLSVLSYERYTVVLQPTQVDVSYFRKAWFCVGGSWLYALFWTLPPLLGWSRYGPEGPGTMCSVQWHLRSPANISYVLCLFIFCLLLPLVVMVYSYGRIWVAVRRQHCAQSHLEAGRINLLTAQRREQHILWMVLSMVSCYMLCWMPYGIIALVATLGRLGPISPAVSVVPSILAKFSTVVNPVIYMFFNNQVR

SEQ ID NO: 11 - protein, 6 aa, synthetic construct
  REGION 1..6: note = ER EXPORT SIGNAL
FXYENE

SEQ ID NO: 12 - skipped

SEQ ID NO: 13 - protein, 5 aa, synthetic construct
  REGION 1..5: note = ER EXPORT SIGNAL
VKESL

SEQ ID NO: 14 - protein, 5 aa, synthetic construct
  REGION 1..5: note = ER EXPORT SIGNAL
VLGSL

SEQ ID NO: 15 - protein, 16 aa, synthetic construct
  REGION 1..16: note = ER EXPORT SIGNAL
NANSFCYENEVALTSK

SEQ ID NO: 16 - protein, 26 aa, synthetic construct
  REGION 1..26: note = MEMBRANE TRAFFICKING SIGNAL
MDYGGALSAVGRELLFVTNPVVVNGS

SEQ ID NO: 17 - protein, 26 aa, synthetic construct
  REGION 1..26: note = MEMBRANE TRAFFICKING SIGNAL
AGHSNSMALFSFSLLWLCSGVLGTEF

SEQ ID NO: 18 - protein, 23 aa, synthetic construct
  REGION 1..23: note = MEMBRANE TRAFFICKING SIGNAL
MGLRALMLWLLAAAGLVRESLQG

SEQ ID NO: 19 - protein, 18 aa, synthetic construct
  REGION 1..18: note = MEMBRANE TRAFFICKING SIGNAL
MRGTPLLLVVSLFSLLQD

SEQ ID NO: 20 - DNA, 864 bp, synthetic construct
  misc_feature 1..864: note = Codon optimized nucleic acid sequence of the MosOpn3
  source 1..864: mol_type = other DNA
atgagcaggaccggccacaccgtggtggccgtgatgctgggcaccatcctgctggccggcgtgttcggcaacagcgtggtgttcctggtgttcgtgaagtacaggagcctgaggacccccatcaacctgatcctgctgaacatcagcctgagcgacatcctggtgtgcgtgttcggcacccccctgagcttcgccgccagcctgaagggcaggtggctgctgggcgagaggggctgcgagtggtacggcttcgccaacagcctgttcggcatcgtgagcctggtgagcctgagcgtgctgagctacgagaggtacaccgtggtgctgcagcccacccaggtggacgtgagctacttcaggaaggcctggttctgcgtgggcggcagctggctgtacgccctgttctggaccctgcctcctctgctgggctggagcaggtacggccccgagggccctggcaccatgtgcagcgtgcagtggcacctgaggagccccgccaacatcagctacgtgctgtgcctgttcatcttctgcctgctgctgcccctggtggtgatggtgtacagctacggcaggatctgggtggccgtgaggaggcagcactgcgcccagagccacctggaggccggcaggatcaacctgctgaccgcccagaggagggagcagcacatcctgtggatggtgctgagcatggtgagctgctacatgctgtgctggatgccctacggcatcatcgccctggtggccaccctgggcaggctgggccccatcagccccgccgtgagcgtggtgcccagcatcctggccaagttcagcaccgtggtgaaccccgtgatctacatgttcttcaacaaccaggtgagg

SEQ ID NO: 21 - DNA, 990 bp, synthetic construct
  misc_feature 1..990: note = Alternative Codon optimized nucleic acid sequence of the MosOpn3
  source 1..990: mol_type = other DNA
atgtacgacgcccccaacgacgtggccagcagcgtggccgactacgaggacctgatggccccctgggcctacaacgccgccgccatcaccctgttcttcatcggcttcttcggcttcttcctgaacctgttcgtgatcgccctgatgagcaaggacatgcagctgtggacccccatgaacatcatcctgttcaacctggtgtgcagcgacttcagcgtgagcatcatcggcaaccccctgaccctgaccagcgccatcagccaccgctggatcttcggccgcaccctgtgcgtggcctacggcttcttcatgagcctgctgggcatcaccagcatcaccaccctgaccgtgctgagctacgagcgctactgcctgatcagccgccccttcagcagccgcaacctgagccgcaagggcgccttcctggccatcttcttcatctggggctacagcttcgccctgaccagcccccccctgttcggctggggcgcctacgtgcaggaggccgccaacatcagctgcagcgtgaactgggagagccagaccaagaacgccaccacctacatcatcttcctgttcgtgttcggcctggtggtgcccctgatcgtgatcgtgtacagctacaccaacatcatcgtgtacatgcgccgcaacagcgcccgcgtgggccgcatcaaccgcgccgagcagcgcgtgaccagcatggtggccgtgatgatcgtggccttcatggtggcctggaccccctacgccatcttcgccctgatcgagcagttcggcccccccgagctgatcggccccggcctggccgtgctgcccgccctgatcgccaagagcagcatctgctacaaccccatcatctacgtgggcatgaacacccagttccgcgccgccttcacccgcgtgcgcaacaagggcggcgtgcccaccgccgaccagaacaccaccaccatgcagcgcgagctgaccaagagcagccgcgacatggtg

SEQ ID NO: 22 - DNA, 864 bp, synthetic construct; identical to SEQ ID NO: 20
  misc_feature 1..864: note = Codon optimized nucleic acid sequence of the PufTMT
  source 1..864: mol_type = other DNA

SEQ ID NO: 23 - DNA, 864 bp, synthetic construct
  misc_feature 1..864: note = Alternative Codon optimized nucleic acid sequence of the PufTMT
  source 1..864: mol_type = other DNA
atgagccgcaccggccacaccgtggtggccgtgatgctgggcaccatcctgctggccggcgtgttcggcaacagcgtggtgttcctggtgttcgtgaagtaccgcagcctgcgcacccccatcaacctgatcctgctgaacatcagcctgagcgacatcctggtgtgcgtgttcggcacccccctgagcttcgccgccagcctgaagggccgctggctgctgggcgagcgcggctgcgagtggtacggcttcgccaacagcctgttcggcatcgtgagcctggtgagcctgagcgtgctgagctacgagcgctacaccgtggtgctgcagcccacccaggtggacgtgagctacttccgcaaggcctggttctgcgtgggcggcagctggctgtacgccctgttctggaccctgccccccctgctgggctggagccgctacggccccgagggccccggcaccatgtgcagcgtgcagtggcacctgcgcagccccgccaacatcagctacgtgctgtgcctgttcatcttctgcctgctgctgcccctggtggtgatggtgtacagctacggccgcatctgggtggccgtgcgccgccagcactgcgcccagagccacctggaggccggccgcatcaacctgctgaccgcccagcgccgcgagcagcacatcctgtggatggtgctgagcatggtgagctgctacatgctgtgctggatgccctacggcatcatcgccctggtggccaccctgggccgcctgggccccatcagccccgccgtgagcgtggtgcccagcatcctggccaagttcagcaccgtggtgaaccccgtgatctacatgttcttcaacaaccaggtgcgc

SEQ ID NO: 24 - protein, 5 aa, synthetic construct
  REGION 1..5: note = repetetive amino acid sequence motif
EAAAK

SEQ ID NO: 25 - protein, 354 aa, synthetic construct
  REGION 1..354: note = PdCO2 aa sequence
MDGENLTIPNPVTELMDTPINSTYFQNLNAETDGGNHYIYNAFTATDYNICAAYLFFIACLGVSLNVLVLVLFIKDRKLRSPNNFLYVSLALGDLLVAVFGTAFKFIITARKTLLREEDGFCKWYGFITYLGGLAALMTLSVIAFVRCLAVLRLGSFTGLTTRMGVAAMAFIWIYSLAFTLAPLLGWNHYIPEGLATWCSIDWLSDETSDKSYVFAIFIFCFLVPVLIIVVSYGLIYDKVRKVAKTGGSVAKAEREVLRMTLLMVSLFMLAWSPYAVICMLASFGPKDLLHPVATVIPAMFAKSSTMYNPLIYVFMNKQFRRSLKVLLGMGVEDLNSESERATGGTATNQVAAT

SEQ ID NO: 26 - DNA, 1062 bp, synthetic construct
  misc_feature 1..1062: note = PdCO2 na sequence
  source 1..1062: mol_type = other DNA
atggacggggagaacttgacgattccaaatcctgtcaccgagttgatggataccccaatcaacagcacgtatttccaaaaccttaatgcggaaacggacggcggaaatcattacatctacaatgccttcacagccaccgattacaacatttgtgcagcttaccttttcttcattgcttgtctgggagtcagtctcaacgttttggtgttggtcctctttatcaaagacagaaagttgaggagccccaataacttcctgtatgtaagtctcgctctcggggatctgttggttgcagttttcggcaccgcatttaagttcattattacagctcgcaaaaccctgttgcgagaagaagacggtttctgcaaatggtacggcttcataacatacctcggcgggctcgcggcactgatgaccctgagcgttattgcttttgttcgttgtctggccgtgctgcgtctcggctccttcacaggtttgacaacgcggatgggcgtggcggcgatggctttcatttggatctactccctggcattcacactcgcgcctctccttgggtggaatcactacataccggaaggtttggccacttggtgtagtatcgactggctttccgatgaaacatccgataagtcctacgtgtttgctattttcatcttctgctttcttgtccctgttctcattatcgtcgtgtcatacgggctgatctacgataaggttcggaaggtagcgaaaacaggcggttctgttgccaaagcagaacgcgaagtactgcgcatgacgctgctgatggttagcctgttcatgcttgcctggagtccctacgctgttatctgcatgctcgctagcttcggccctaaagacctgctccatccagtggccacagtgattcctgccatgtttgcaaagtcttctacgatgtataaccctcttatctatgtgttcatgaacaaacaattcaggcggagccttaaagttcttctcggaatgggagtagaggacctgaactccgagagtgagagagcaactggtggtacagccacgaatcaagtcgccgcgacg

SEQ ID NO: 27-28 - skipped (2 entries)

SEQ ID NO: 29 - protein, 343 aa, synthetic construct
  REGION 1..343: note = LcPP aa sequence
MENLTSLDLLPNGEVPLMPRYGFTILAVIMAVFTIASLVLNSTVVIVTLRHRQLRHPLNFSLVNLAVADLGVTVFGASLVVETNAVGYFNLGRVGCVIEGFAVAFFGIAALCTIAVIAVDRFVVVCKPLGTLMFTRRHALLGIAWAWLWSFVWNTPPLFGWGSYELEGVRTSCAPDWYSRDPANVSYITSYFAFCFAIPFLVIVVAYGRLMWTLHQVAKLGMGESGSTAKAEAQVSRMVVVMVVAFLVCWLPYALFAMIVVTKPDVYIDPVIATLPMYLTKTSTVYNPIIYIFMNRQFRDCAVPFLLCGRNPWAEPSSESATAASTSATSVTLASAPGQVSPS

SEQ ID NO: 30 - DNA, 1029 bp, synthetic construct
  misc_feature 1..1029: note = LcPP na sequence
  source 1..1029: mol_type = other DNA
atggaaaatctgacttccctggatctgctgcccaacggagaggtcccactgatgccccggtacggctttactattctggctgtgattatggccgtgttcaccatcgcaagtctggtcctgaactcaactgtggtcattgtgaccctgcgacaccgacagctgaggcatcctctgaacttttccctggtgaatctggctgtcgcagacctgggcgtgacagtcttcggagcttctctggtggtcgagactaacgcagtggggtactttaatctgggacgcgtggggtgcgtcatcgaagggttcgccgtggctttctttggcattgccgctctgtgcaccatcgctgtgattgcagtcgatcgatttgtggtggtgtgcaagcccctgggaaccctgatgttcacaaggagacacgcactgctgggaatcgcatgggcatggctgtggagcttcgtgtggaacacaccccctctgttcggctggggaagctacgagctggaaggagtgagaactagctgcgctcctgactggtattcccgggaccccgccaacgtgagctacatcacatcttatttcgcattttgtttcgccatccccttcctggtcatcgtcgtggcttacggccggctgatgtggactctgcatcaggtggccaagctggggatgggcgagtctggaagtaccgctaaagcagaagcccaggtgagtcgcatggtcgtggtcatggtggtcgcctttctggtctgttggctgccctatgccctgttcgctatgatcgtggtcaccaagcctgacgtgtacatcgatccagtcattgccacactgcccatgtatctgaccaaaacaagcaccgtgtacaaccccatcatctacatcttcatgaatcgacagttcagggactgcgccgtgcctttcctgctgtgcggcaggaatccctgggcagagcccagctccgaatctgccacagcagcctcaaccagcgccacaagtgtgactctggcttcagcaccaggacaggtctccccatcc

SEQ ID NO: 31-32 - skipped (2 entries)

SEQ ID NO: 33 - protein, 342 aa, synthetic construct
  REGION 1..342: note = medakaTMT1A aa sequence
MLVSNVSLGGCAEFNSALCAGAGEEHLGGGSYRTTLTPTGHLIVAVCLGFIGTFGLVNNLLVLVLFCRYKILRSPINLLLINISISDLLVCVLGTPFSFAASTQGRWLIGEGGCVWYGFANSLCGIVSLISLAVLSYERYSTMMTPAEADSSNYRKISLGIILSWGYSLLWTLPPLFGWSHYGPEGPGTTCSVDWTAKTANNISYIICLFVFCLIVPFMVIVFCYGKLLYAIKQVSGINVSVSRKREQRVLFMVVIMVICYLLCWLPYGIMALLATFGPPDLVTPEASIIPSVLAKTSTAINPVIYVFMNKQFFRCFQAMLRCKAPLRGSSARSSSKVATKA

SEQ ID NO: 34 - DNA, 1026 bp, synthetic construct
  misc_feature 1..1026: note = medakaTMT1A na sequence
  source 1..1026: mol_type = other DNA
atgctagtcagtaatgtaagcctgggtgggtgcgccgaattcaattccgctctttgtgcgggcgctggagaagaacacctcggcggtggttcatatcggaccacgctaaccccgactggccatctcattgtcgctgtgtgccttgggttcattggcactttcgggcttgtaaataatttgcttgtcttggtcctattctgtcgttataagattctgcggagccctatcaatcttctacttattaatatcagtatttccgacctgctcgtttgcgtgctgggcacccctttctcctttgctgctagtacacaaggcaggtggttgattggcgagggcggatgtgtatggtacggatttgcgaacagcctgtgtgggatcgttagcctaatttcccttgctgtcctttcttatgaacgttactctactatgatgacccccgcggaagccgactcaagtaattaccggaaaataagtctcggtatcatcctctcctggggctatagtttgctctggacgttgccccctttgtttggctggagtcattacgggcccgaaggaccaggaaccacctgtagcgtcgattggaccgccaagaccgccaataacattagctatattatctgcctgtttgtgttctgtcttatcgtgccgtttatggtgattgtattttgctatggtaaactgctgtacgctattaaacaagtgagcggaattaatgtcagtgtaagtaggaaacgagaacaacgcgtgctctttatggtggtcattatggtcatatgctacctgctttgttggctcccttacggcattatggcccttctcgcaacgtttggacccccagacctcgtcaccccagaagcctctatcataccctcagttctcgcgaagactagtaccgctataaatccagttatctatgtctttatgaataagcagttcttcaggtgtttccaagcaatgcttaggtgtaaagctccactgcgcgggagctcagcaaggtccagctccaaagttgctacaaaagct

SEQ ID NO: 35-36 - skipped (2 entries)

SEQ ID NO: 37 - protein, 341 aa, synthetic construct
  REGION 1..341: note = zPP1 aa sequence
MHEEMESETSTAASGSIAEVMPRTGYTILAVIIGVFSVCGVILNVTVITVTLKYKQLRQPLNFALVNLAVADLGCAVFGGLPTVVTNAMGYFSLGRVGCVLEGFAVAFFGIAALCSVAVIALERCMVVCRPVGSISFQTRHAVFGVAVSWVWSFIWNTPPLFGWGRFELEGVRTSCAPDWYSRDLANVSFIVCYFLLCFALPFSVIVYSYTRLLWTLRQVSRLQVCEGGSAARAEAQVSCMVVVMILAFLLTWLPYASFALCVILIPELYIDPVIATVPMYLTKSSTVFNPIIYIFMNRQFRDRALPFLLCGRNPWAAEAEEEEEETTVSSVSRSTSVSPA

SEQ ID NO: 38 - DNA, 1023 bp, synthetic construct
  misc_feature 1..1023: note = zPP1 na sequence
  source 1..1023: mol_type = other DNA
atgcacgaggaaatggaatcagaaacatctacagcggcctctgggagcattgctgaggtgatgccccgcactggatataccatacttgcagtgattatcggggtcttttcagtctgcggtgtaatactgaacgtaaccgttattaccgtcacacttaaatacaaacagttgcgtcaacctctgaattttgcgctcgttaatctggccgtagcagacctgggatgtgcggtctttggtggccttccaacggttgtcacaaatgccatgggatatttctctttgggccgcgtcggctgcgtactcgaaggattcgctgttgctttcttcggtatcgctgcattgtgcagcgtcgccgtaattgctctcgaacggtgcatggtggtctgcagacccgtggggtcaatcagtttccaaaccagacatgccgtgtttggagttgccgtgagctgggtatggtcatttatctggaacacaccccctctcttcggttggggcaggtttgaacttgagggagtgcggacgagttgcgctccagattggtacagtagagatcttgccaatgtcagctttatagtttgttacttcttgctgtgttttgccttgcctttctctgtgattgtatacagctacactcgtttgctctggacactccgacaagtatctcggctgcaagtatgtgaaggcggttctgcggcacgcgctgaagctcaagtatcctgcatggtagtggtcatgatactcgcttttctcctcacttggctgccatatgcaagtttcgctttgtgtgttatcctcatacctgagctctatatcgaccctgtcatcgccacggtcccaatgtatctcacaaaatcctcaactgtcttcaaccctatcatctatatatttatgaatcggcagtttagggaccgtgctctcccgtttctgctgtgcgggagaaatccatgggctgcggaagccgaggaagaagaagaggaaacaaccgtgtcaagcgtcagcaggtccactagcgtaagccccgcg

SEQ ID NO: 39-40 - skipped (2 entries)

SEQ ID NO: 41 - protein, 336 aa, synthetic construct
  REGION 1..336: note = pPP2 aa sequence
MKPSAFYLNASLYLGPQGEPPLPRSGFIALSVIMALLTGPAIVLNATVIIVSLMHKQLRQPLNYALVNMAVADLGTAMTGGLLSVVNNAQGYFSLGRTGCVLEGFAVSLCGIASLCTVALIAVERMFVICKPLGQMQFQKQHALGGIALAWLWSLTWNLPPLFGWGRYELEGVGTSCAPDWHSREPQNVSYVLAYFTVCFAAPFVIILVSYSKLMWTLHKVTKMACMEGGAVAKSEMTVAYMVILMVVTFLISWLPYAGLSMLVVLSPDVKIHPLVGTVPVYLAKSSTVYNPIIYIYLNKQFRKYAVPFLLCGRELEMEDELSMTTVETSNRVSPA

SEQ ID NO: 42 - DNA, 1008 bp, synthetic construct
  misc_feature 1..1008: note = pPP2 na sequence
  source 1..1008: mol_type = other DNA
atgaagccttctgcattctacctaaatgcaagtctgtacctcgggccacagggagagcccccactgcctcggagtggctttattgctctgtctgtgatcatggctttgctcacaggtccagccatagtcttgaacgctacagtgataatcgtgtccctcatgcataaacaactaaggcagcctttgaattatgcgctcgttaatatggcagtggccgatcttggtacagctatgacgggcggcctgctgtctgtggtgaataatgcgcaaggatactttagcctgggcagaacaggttgcgtactggaaggttttgctgttagtctctgcggcattgcatctctgtgtacggtggcactgatcgccgtcgaacgcatgttcgtcatttgtaaacctctgggccaaatgcaattccaaaagcagcacgcattgggcggcatcgccctggcttggctttggtctctgacatggaatctcccacccctctttgggtggggcagatatgaacttgagggtgtgggcacctcatgcgcgcccgactggcatagccgggagccacaaaatgtatcatacgtccttgcttatttcactgtgtgttttgccgcaccatttgttatcattctggtctcatatagcaaactgatgtggactctgcacaaagtcactaaaatggcttgtatggaaggtggagcagtcgcaaagtctgaaatgaccgtggcctacatggttatcctcatggtagttacattcttgatcagctggctcccctacgccgggctcagcatgctcgtggttctcagcccggatgtgaagatacacccgcttgtgggtactgttcctgtgtacctggctaaatcctctacagtatacaatcccatcatttacatttatttgaacaaacaattccgcaagtacgccgtcccatttctgctctgcggccgggaactcgaaatggaagacgagctttccatgactacagtagaaactagcaatagagttagccctgcc

SEQ ID NO: 43-44 - skipped (2 entries)